(19) United States
(12) Patent Application Publication
(10) Pub. No.: US 2019/0062487 A1
(43) Pub. Date: Feb. 28, 2019

(54) LIQUID CRYSTALLINE ELASTOMER PRECURSOR AND LIQUID CRYSTALLINE ELASTOMER

(71) Applicant: TOYO TIRE & RUBBER CO., LTD., Itami-shi, Hyogo (JP)

(72) Inventor: Seiji Iseki, Itami-shi (JP)

(73) Assignee: TOYO TIRE & RUBBER CO., LTD., Itami-shi, Hyogo (JP)

(21) Appl. No.: 16/070,555

(22) PCT Filed: Feb. 8, 2017
(86) PCT No.: PCT/JP2017/004468

§ 371 Date: Jul. 17, 2018

(30) Foreign Application Priority Data
Apr. 8, 2016 (JP) 2016-078409

(51) Int. Cl. C08G 18/32; C08G 18/73; C07C 69/67; C07C 69/78

(52) U.S. Cl. CPC C08G 18/3221; C08G 18/73; C08G 2250/00; C07C 69/78; C07C 69/67

(57) ABSTRACT

Provided is a novel liquid crystalline elastomer that can reversibly undergo phase transition between a liquid crystalline phase and an isotropic phase thereof in a relatively low temperature region. A liquid crystalline elastomer precursor comprising a mesogenic group to which an oxide compound is attached, wherein the liquid crystalline elastomer precursor has a molecular moiety excluding the mesogenic group, the molecular moiety having at least one ester bond and at least two active hydrogen groups. A liquid crystalline elastomer comprising the liquid crystalline elastomer precursor cross-linked by a trifunctional or higher-functional isocyanate compound and/or polyol compound, wherein the liquid crystalline elastomer has a molecular moiety excluding the mesogenic group, the molecular moiety having at least one ester bond, and the liquid crystalline elastomer reversibly changes a state thereof between a liquid crystalline phase and isotropic phase thereof in response to a change in temperature thereof.

### TECHNICAL FIELD

The present invention relates to a liquid crystalline elastomer precursor containing a mesogenic group to which an oxide compound is attached, and a liquid crystalline elastomer synthesized from the liquid crystalline elastomer precursor.

### BACKGROUND ART

Liquid crystalline polymers that have a mesogenic group in their molecular structure change their physical properties with a change in the degree of alignment of the liquid crystal (mesogenic group). Attention has been focused on such nature, and attempts have been made to use liquid crystalline polymers as elastomers in various applications.

For example, a liquid crystalline polyurethane (liquid crystalline polymer) that is obtained by addition polymerization of a diol component and a diisocyanate component, and has thermotropic liquid crystallinity in at least a predetermined macromolecular region, has been disclosed (see, for example, Patent Document 1).

### CITATION LIST

### Patent Literature

Patent Document 1: Japanese Unexamined Patent Application Publication No. H05-170860

### SUMMARY OF INVENTION

### Technical Problem

A liquid crystal elastomer that can be incorporated into industrial products for practical use is required to change its dynamic physical properties or displacement amount greatly in response to a change in temperature, etc., of an external environment while maintaining at least a predetermined strength (durability).

In this regard, Patent Document 1 discloses a liquid crystalline polymer that contains a mesogen and therefore can undergo alignment (i.e., phase transition), and indicates that the phase transition temperature (Ti) of that liquid crystalline polymer is 200° C. or more. Therefore, it is considered that it is difficult to use the liquid crystalline polymer of Patent Document 1 as a material for general industrial products.

With the above circumstances in mind, the present invention has been made. It is an object of the present invention to provide a novel liquid crystalline elastomer that can reversibly undergo phase transition between a liquid crystalline phase and isotropic phase thereof even in a relatively low temperature region, and a liquid crystalline elastomer precursor that is a raw material for the liquid crystalline elastomer.

### Solution to Problem

To achieve the above object, the present invention provides a liquid crystalline elastomer precursor comprising:

a mesogenic group to which an oxide compound is attached,

wherein

the liquid crystalline elastomer precursor has a molecular moiety excluding the mesogenic group, the molecular moiety having at least one ester bond and at least two active hydrogen groups.

In general, it is known that the molecular structure of a polymer material has much influence on its physical properties. In a liquid crystalline elastomer obtained by cross-linking a liquid crystalline elastomer precursor, it is important to understand the correlation between the molecular structure and physical properties of the liquid crystalline elastomer in the design of the liquid crystalline elastomer. With this in mind, the present inventors have focused on the fact that the phase transition temperature (Ti) of a liquid crystalline elastomer varies depending on the molecular structure of a liquid crystalline elastomer precursor that is a raw material for the liquid crystalline elastomer, in order to develop a novel liquid crystalline elastomer. The present inventors have made attempts to find a liquid crystalline elastomer that achieves the object of the present invention by changing the molecular structure of a liquid crystalline elastomer precursor.

The liquid crystalline elastomer precursor having the above feature contains a mesogenic group to which an oxide compound is attached, and has at least one ester bond and at least two active hydrogen group in a molecular moiety thereof excluding the mesogenic group. In a liquid crystalline elastomer precursor that satisfies such conditions, the oxide compound acts to reduce the thermal stability of the mesogenic group contained in the liquid crystalline elastomer precursor. Therefore, the temperature at which liquid crystallinity occurs in the liquid crystalline elastomer precursor is reduced. In this case, the temperature at which liquid crystallinity occurs in a liquid crystalline elastomer synthesized from the liquid crystalline elastomer precursor is also reduced. Therefore, such a liquid crystalline elastomer can be moded at or near room temperature without a solvent. In addition, the mesogenic group contained in the liquid crystalline elastomer precursor allows a liquid crystalline elastomer obtained from the liquid crystalline elastomer precursor to have both liquid crystallinity and elasticity. In particular, the mesogenic group allows the liquid crystalline elastomer precursor to be used as a raw material for the thermoresponsive liquid crystalline elastomer that reversibly changes its state in response to a change in temperature thereof. Furthermore, the at least one ester bond in the molecular moiety excluding the mesogenic group allows introduction of spacers having various structures to the liquid crystalline elastomer precursor through the ester bond. In this case, by changing the molecular structure of the spacer, the transition temperature (Ti) of a liquid crystalline elastomer obtained from the precursor can be adjusted.

In the liquid crystalline elastomer precursor of the present invention,

the liquid crystalline elastomer precursor is preferably represented by:

Z₁-E₁-D₁-C₁-B₁-Y₁-A₁-X-A₂-Y₂-B₂-C₂-D₂-E₂-Z₂ (1)

where X is a portion of the molecular structure of the mesogenic group and represents a single bond that forms a portion of binding groups adjacent thereto, —N═N—, —CO—, —CH═N—, —CO—O—, —CH₂—, —CH═CH—, or —CO—NH—, A₁ and A₂ are the same or different and independently represent a cycloalkane having 3-8 carbon atoms, a benzene ring, naphthalene, biphenyl, or a heterocyclic compound thereof, optionally partially substituted with —Br, —Cl, or —CH₃, Y₁ and Y₂ are the same or different and independently represent a single bond that forms a portion of binding groups adjacent thereto, —O—, —CO—, —S—, —S—, or —Te—, B₁ and B₂ are the same or different and independently represent a single bond that forms a portion of binding groups adjacent thereto, or —(CH₂)ₘ— where m represents an integer of 1-20, C₁ and C₂ are a binding group derived from the oxide compound, are the same or different, and independently represent —((CₙH₂ₙ)O)ₚ— where n represents an integer of 2-4 and p represents an integer of 1-5, or —(((C₆H₅)C₂H₃)O)_{q}— where q represents an integer of 1-5, at least one of D₁ and D₂ represents the ester bond, E₁ and E₂ are the same or different and independently represent a single bond that forms a portion of binding groups adjacent thereto, provided that the adjacent binding groups are not the ester bond, —CO—, —(CH₂)ᵣCO— where r represents an integer of 1-8, or —(C₆H₄)CO—, Z₁ and Z₂ are a terminal group having the active hydrogen group, are the same or different, and independently represent —OH, —SH, —COOH, —CHO, or —O—CH(OH)—CH₂OH.

In the liquid crystalline elastomer precursor having the above feature, the molecular structure thereof contains an appropriate binding group and functional group. A liquid crystalline elastomer obtained from the liquid crystalline elastomer precursor as a raw material has sufficient strength and durability and is practically useful.

In the liquid crystalline elastomer precursor of the present invention, preferably, the X represents a single bond that forms a portion of binding groups adjacent thereto, CH═N—, or —CO—O—, the A₁ and the A₂ are the same and represent a benzene ring, the Y₁ and the Y₂ are the same and represent —O—, the B₁ and the B₂ are the same and represent a single bond that forms a portion of binding groups adjacent thereto, or —(CH₂)₆—, the C₁ and the C₂ are the same and represent —((CₙH₂ₙ)O)ₚ— where n represents an integer of 2-4 and p represents an integer of 1-4, or —((C₆H₅)C₂H₃)O—, the D₁ represents the ester bond, the D₂ represents a single bond that forms a portion of binding groups adjacent thereto, the E₁ represents —(CH₂)ᵣCO— where r represents an integer of 3 or 4, or —(C₆H₄)CO—, the E₂ represents a single bond that forms a portion of binding groups adjacent thereto, and the Z₁ and the Z₂ are the same and represent —OH.

In the liquid crystalline elastomer precursor having the above feature, a more appropriate binding group and functional group are provided in the molecular structure thereof. Therefore, a liquid crystalline elastomer obtained from the liquid crystalline elastomer precursor as a raw material has excellent thermoresponsiveness in addition to sufficient strength and durability.

To achieve the above object, the present invention provides a liquid crystalline elastomer comprising:

the liquid crystalline elastomer precursor having any one of the above features cross-linked by a trifunctional or higher-functional isocyanate compound and/or polyol compound, wherein

the liquid crystalline elastomer has a molecular moiety excluding the mesogenic group, the molecular moiety having at least one ester bond, and

the liquid crystalline elastomer reversibly changes a state thereof between a liquid crystalline phase and isotropic phase thereof in response to a change in temperature thereof.

In the liquid crystalline elastomer having the above feature, the liquid crystalline elastomer precursor is cross-linked by a trifunctional or higher-functional isocyanate compound and/or polyol compound, and at least one ester bond is provided in the molecular moiety excluding the mesogenic group. The use of the isocyanate compound and/or polyol compound having at least three reactive functional groups as a cross-linking agent allows the liquid crystalline elastomer to have a dense molecular structure, and thereby provide at least a predetermined strength when used as a material. Therefore, the durability of the liquid crystalline elastomer can be improved while the thermoresponsiveness thereof is maintained when the liquid crystalline elastomer undergoes phase transition from the liquid crystalline phase to the isotropic phase. Furthermore, the at least one ester bond in the molecular moiety excluding the mesogenic group allows introduction of spacers having various structures to the liquid crystalline elastomer through the ester bond. In this case, by changing the molecular structure of the spacer, the transition temperature (Ti) of the liquid crystalline elastomer can be adjusted. In addition, the liquid crystalline elastomer reversibly changes its state between the liquid crystalline phase and the isotropic phase in response to a change in temperature thereof, and therefore, can be particularly used as a thermoresponsive liquid crystalline elastomer that reversibly expands and contracts in response to a change in temperature thereof.

In the liquid crystalline elastomer of the present invention,

a phase transition temperature (Ti) that is a boundary between the liquid crystalline phase and the isotropic phase is preferably −10 to 100° C.

The phase transition temperature (Ti) of the liquid crystalline elastomer having the above feature is between −10 and 100° C. Therefore, the liquid crystalline elastomer can change its state in a relatively low temperature range including room temperature or human body temperature. Therefore, the liquid crystalline elastomer is convenient and practically useful in daily life.

### DESCRIPTION OF EMBODIMENTS

Embodiments of a liquid crystalline elastomer precursor and liquid crystalline elastomer according to the present invention will now be described. Note that the present invention is in no way limited to features described in the embodiments below.

### Structure of Liquid Crystalline Elastomer Precursor

The liquid crystalline elastomer precursor of the present invention is a liquid crystalline compound containing a mesogenic group to which an oxide compound is attached.

The oxide compound may be an alkylene oxide and/or a styrene oxide, etc. Examples of the alkylene oxide include ethylene oxide, propylene oxide, and butylene oxide. The above alkylene oxides may be used alone or in combination. The styrene oxide may have a substituent, such as an alkyl group, an alkoxyl group, or a halogen, on the benzene ring. The alkylene oxide may be a combination of the above alkylene oxides and the above styrene oxides. In the liquid crystalline elastomer precursor, the oxide compound acts to reduce the thermal stability of the mesogenic group contained in the liquid crystalline elastomer precursor, and thereby reduce the temperature at which the liquid crystallinity of the liquid crystalline elastomer precursor occurs. In this case, the temperature at which the liquid crystallinity of the liquid crystalline elastomer synthesized from the liquid crystalline elastomer precursor occurs may also decrease, and therefore, the liquid crystalline elastomer can be molded at or near room temperature without a solvent. The blended amount of the alkylene oxide and/or styrene oxide is adjusted to 1-10 mol, preferably 2-8 mol, with respect to 1 mol of the mesogenic group-containing compound. If the number of moles of the alkylene oxide and/or styrene oxide attached is less than 1 mol, it is difficult to sufficiently lower the temperature range within which the liquid crystallinity of the liquid crystalline elastomer occur, and therefore, it is difficult to continuously mold the liquid crystalline elastomer while the raw materials are reacted and cured without a solvent and with liquid crystallinity occurring. If the number of moles of the alkylene oxide and/or styrene oxide attached exceeds 10 mol, the liquid crystallinity of the liquid crystalline elastomer is less likely to occur.

The liquid crystalline elastomer precursor of the present invention has at least one ester bond and at least two active hydrogen groups in a molecular moiety thereof excluding the mesogenic group. The mesogenic group contained in the liquid crystalline elastomer precursor allows a liquid crystalline elastomer obtained from the liquid crystalline elastomer precursor to have both liquid crystallinity and elasticity. In particular, the mesogenic group allows the liquid crystalline elastomer precursor to be used as a raw material for a liquid crystalline elastomer that reversibly changes its state in response to a change in temperature thereof. Furthermore, the at least one ester bond in the molecular moiety excluding the mesogenic group allows introduction of spacers having various structures to the liquid crystalline elastomer precursor through the ester bond. In this case, by changing the molecular structure of the spacer, the transition temperature (Ti) of a liquid crystalline elastomer obtained from the precursor can be adjusted.

The liquid crystalline elastomer precursor is, for example, a compound represented by a general formula (1) below.

Z₁-E₁-D₁-C₁-B₁-Y₁-A₁-X-A₂-Y₂-B₂-C₂-D₂-E₂-Z₂ (1)

In the general formula (1), preferably, X is a portion of the molecular structure of the mesogenic group and represents a single bond that forms a portion of binding groups adjacent thereto, —N═N—, —CO—, —CH═N—, —CO—O—, —CH₂—, —CH═CH—, or —CO—NH—, A₁ and A₂ are the same or different and independently represent a cycloalkane having 3-8 carbon atoms, a benzene ring, naphthalene, biphenyl, or a heterocyclic compound thereof, optionally partially substituted with —Br, —Cl, or —CH₃, Y₁ and Y₂ are the same or different and independently represent a single bond that forms a portion of binding groups adjacent thereto, —O—, —CO—, —S—, —Se—, or —Te—, B₁ and B₂ are the same or different and independently represent a single bond that forms a portion of binding groups adjacent thereto, or —(CH₂)ₘ— where m represents an integer of 1-20, C₁ and C₂ are a binding group derived from the oxide compound, are the same or different, and independently represent —((CₙH₂ₙ)O)ₚ— where n represents an integer of 2-4 and p represents an integer of 1-5, or —(((C₆H₅)C₂H₃)O)_{q}— where q represents an integer of 1-5, at least one of D₁ and D₂ represents the ester bond, E₁ and E₂ are the same or different and independently represent a single bond that forms a portion of binding groups adjacent thereto, provided that the adjacent binding groups are not the ester bond, —CO—, —(CH₂)ᵣCO— where r represents an integer of 1-8, or —(C₆H₄)CO—, Z₁ and Z₂ are a terminal group having the active hydrogen group, are the same or different, and independently represent —OH, —SH, —COOH, —CHO, or —O—CH(OH)—CH₂OH. Note that the term “single bond that forms a portion of binding groups adjacent thereto” means that the single bond is shared by the binding groups adjacent thereto. For example, in the general formula (1), if C₁ is —O(C₃H₆)—, Y₁ is —O—, and B₁ is a single bond that forms a portion of binding groups adjacent thereto, the moiety C₁—B₁—Y₁ is —O(C₃H₆)—O—, and the single bond is shared by —O(C₃H₆)— and —O— on the opposite sides of the single bond. The liquid crystalline elastomer precursor having this structure has appropriate binding groups and functional groups in the molecular structure thereof. Therefore, a liquid crystalline elastomer obtained from the liquid crystalline elastomer precursor as a raw material has sufficient strength and durability and is practically useful.

In the general formula (1), more preferably, X represents a single bond that forms a portion of binding groups adjacent thereto, —CH═N—, or —CO—O—, A₁ and A₂ are the same and represent a benzene ring, Y₁ and Y₂ are the same and represent —O—, B₁ and B₂ are the same and represent a single bond that forms a portion of binding groups adjacent thereto, or —(CH₂)₆—, C₁ and C₂ are the same and represent —((CₙH₂ₙ)O)ₚ— where n represents an integer of 2-4 and p represents an integer of 1-4, or —((C₆H₅)C₂H₃)O—, D₁ represents the ester bond, D₂ represents a single bond that forms a portion of binding groups adjacent thereto, E₁ represents —(CH₂)ᵣCO— where r represents an integer of 3 or 4, or —(C₆H₄)CO—, E₂ represents a single bond that forms a portion of binding groups adjacent thereto, and Z₁ and Z₂ are the same and represent —OH. In the liquid crystalline elastomer precursor having this structure, a more appropriate binding group and functional group are provided in the molecular structure thereof. Therefore, a liquid crystalline elastomer obtained from the liquid crystalline elastomer precursor as a raw material has excellent thermoresponsiveness in addition to sufficient strength and durability.

### Structure of Liquid Crystalline Elastomer

The liquid crystalline elastomer is obtained by cross-linking the liquid crystalline elastomer precursor using a trifunctional or higher-functional isocyanate compound and/or polyol compound. That is, the isocyanate compound and polyol compound are used as a cross-linking agent. The use of the isocyanate compound and/or polyol compound having at least three reactive functional groups as a cross-linking agent allows the liquid crystalline elastomer to have a dense molecular structure, and thereby provide at least a predetermined strength when used as a material. Therefore, the durability of the liquid crystalline elastomer can be improved while the thermoresponsiveness thereof is maintained when the liquid crystalline elastomer undergoes phase transition from the liquid crystalline phase to the isotropic phase.

Examples of the isocyanate compound having at least three reactive functional groups include: triisocyanates, such as triphenylmethane triisocyanate, tris(isocyanatophenyl)thiophosphate, lysine ester triisocyanate, 1,3,6-hexamethylene triisocyanate, 1,6,11-undecane triisocyanate, 1,8-diisocyanate-4-isocyanatomethyl octane, and bicycloheptane triisocyanate; and tetraisocyanates, such as tetraisocyanatesilane. These trifunctional or higher-functional isocyanate compounds may be used alone or in combination.

Examples of the polyol compound having at least three reactive functional groups include: high-molecular-weight polyols having three or more hydroxy groups (molecular weight: 400 or more), such as polyether polyols, polyester polyols, polycarbonate polyols, and polyester polycarbonate polyols; and low-molecular-weight polyols, such as trimethylolpropane, glycerol, 1,2,6-hexanetriol, meso-erythritol, pentaerythritol, tetramethylol cyclohexane, methyl glucoside, sorbitol, mannitol, dulcitol, sucrose, 2,2,6,6-tetrakis(hydroxymethyl)cyclohexanol, and triethanolamine. These polyols may be used alone or in combination.

The blended amount of the cross-linking agent is adjusted to 0.1 to 20 parts by weight, preferably 0.2 to 18 parts by weight, with respect to a total of 100 parts by weight of all the raw materials (the liquid crystalline elastomer precursor and the cross-linking agent). When the blended amount of the cross-linking agent is within such a range, the mesogenic group in the liquid crystalline elastomer can move to an appropriate extent, resulting in well-balanced thermoresponsiveness and liquid crystallinity. If the blended amount of the cross-linking agent is less than 0.1 parts by weight, the liquid crystalline elastomer is not sufficiently cured, and therefore, the liquid crystalline elastomer itself is likely to flow, resulting in a failure to achieve thermoresponsiveness. If the blended amount of the cross-linking agent exceeds 20 parts by weight, the liquid crystalline elastomer has an excessively high cross-link density, and therefore, the alignment of the mesogenic group is likely to be inhibited, resulting in difficulty in achieving liquid crystallinity and therefore a failure to achieve thermoresponsiveness.

The liquid crystalline elastomer of the present invention has at least one ester bond in a molecular moiety thereof excluding the mesogenic group. As a result, spacers having various molecular structures can be introduced to the liquid crystalline elastomer precursor through the ester bond. By changing the molecular structure of the spacer, the transition temperature (Ti) of the liquid crystalline elastomer can be adjusted. In addition, the liquid crystalline elastomer reversibly transitions between the liquid crystalline phase and the isotropic phase in response to a change in temperature thereof, and therefore, can be particularly used as a thermoresponsive liquid crystalline elastomer that reversibly expands and contracts in response to a change in temperature thereof.

The liquid crystalline elastomer of the present invention may contain, in addition to the main component, a small amount of a sub-component or sub-components (e.g., other polymers, low-molecular-weight substances, fillers, etc.) and a small three-dimensional structure or structures (e.g., bubbles, voids, etc.). As used herein, the “matrix” means the main component of the material.

The liquid crystalline elastomer is produced by, for example, the following reaction scheme. Initially, the mesogenic group-containing compound and the alkylene oxide and/or styrene oxide are reacted together to formulate an adduct between the mesogenic group-containing compound and the alkylene oxide and/or styrene oxide (the adduct is hereinafter referred to as the “mesogenic diol”). The mesogenic diol thus formulated is reacted with a dicarboxylic acid or a dicarboxylic acid derivative to formulate a liquid crystalline elastomer precursor having an ester bond. The trifunctional or higher-functional isocyanate compound and/or polyol compound are added as a cross-linking agent to the liquid crystalline elastomer precursor, following by mixing while heating, to obtain a half-cured liquid crystalline compound (pre-polymer). When the half-cured liquid crystalline compound is cured under appropriate conditions, the liquid crystalline compound is cured while being polymerized, to form the liquid crystalline elastomer. The liquid crystalline elastomer is formed into fiber, film, foam, etc., depending on the application thereof. In this case, when the liquid crystalline elastomer is formed while being stretched at a temperature that is more than or equal to the glass transition temperature (Tg) and less than or equal to the phase transition temperature (Ti) (i.e., a temperature at which liquid crystallinity occurs), the mesogenic group contained in the liquid crystalline elastomer is moved and highly aligned in the stretching direction. When the stretched liquid crystalline elastomer is cured, the thermoresponsive material having both liquid crystallinity and elasticity is completed. When the thermoresponsive material is heated, the mesogenic group in the liquid crystalline elastomer, which is normally aligned in the stretching direction, is disaligned (irregularly oriented), so that the thermoresponsive material contracts in the stretching direction. When the heat is removed, the alignment of the mesogenic group is restored, so that the thermoresponsive material expands in the stretching direction, which is a remarkable thermoresponsive behavior.

Incidentally, the alignment properties of the liquid crystalline elastomer can be evaluated by the degree of alignment of the mesogenic group. The mesogenic group having a higher degree of alignment is more highly aligned in a uniaxial direction. The degree of alignment is calculated on the basis of the following calculation expression having, as parameters, the antisymmetric-stretching-vibrational absorbance (0°, 90°) of an aromatic ether and the symmetric-bending-vibrational absorbance (0°, 90°) of a methyl group, which are measured by one-time attenuated total reflection (ATR) using a Fourier-transform infrared spectrometer (FT-IR).

Degree of alignment=(A−B)/(A+2B)

A: (the antisymmetric-stretching-vibrational absorbance of an aromatic ether as measured at 0°)/(the symmetric-bending-vibrational absorbance of a methyl group as measured at 0°)

B: (the antisymmetric-stretching-vibrational absorbance of an aromatic ether as measured at 90°)/(the symmetric-bending-vibrational absorbance of a methyl group as measured at 90°)

To cause significant elasticity to occur in the liquid crystalline elastomer, the degree of alignment of the liquid crystalline elastomer is preferably 0.05 or more, more preferably 0.1 or more.

The liquid crystalline elastomer obtained by the above reaction scheme can be directly used as the matrix of a thermoresponsive material. Alternatively, a small amount of a sub-component or sub-components may be added to the liquid crystalline elastomer, or bubbles may be dispersed in the liquid crystalline elastomer. Examples of the sub-components that can be added to the liquid crystalline elastomer include organic fillers, inorganic fillers, reinforcing agents, thickeners, release agents, excipients, coupling agents, flame retardants, flame-resistant agents, pigments, colorants, deodorants, antimicrobial agents, antifungal agents, antistatic agents, UV protective agents, and surfactants. As a sub-component, another polymer or a low-molecular-weight substance can be added. The liquid crystalline elastomer additionally containing sub-components has the functions of the sub-components, and therefore, can be used in more applications.

### Phase Transition Temperature (Ti) of Liquid Crystalline Elastomer

In order to allow the liquid crystalline elastomer to be used in a temperature region including room temperature, a liquid crystalline polymer that has an appropriate phase transition temperature (Ti) needs to be selected as the matrix of the liquid crystalline elastomer. In the present invention, the liquid crystalline polymer preferably has a phase transition temperature (Ti) −10 to 100° C. Furthermore, the difference between the phase transition temperature (Ti) and the glass transition temperature (Tg) is preferably 20° C. or more, more preferably 25° C. or more. Such a liquid crystalline elastomer can change its state in a relatively low temperature range including room temperature or human body temperature. Therefore, the liquid crystalline elastomer is convenient and practically useful in daily life.

### EXAMPLES

To verify the usefulness of the liquid crystalline elastomer precursor and liquid crystalline elastomer of the present invention, liquid crystalline elastomer precursors were produced from varied amounts of raw materials or different raw materials, and liquid crystalline elastomers were produced by cross-linking the liquid crystalline elastomer precursors, and characteristics thereof were evaluated. Examples of the liquid crystalline elastomer will now be described.

### Synthesis of Liquid Crystalline Elastomer

A liquid crystalline elastomer precursor was synthesized, and a liquid crystalline elastomer was synthesized from that liquid crystalline elastomer precursor (Examples 1-12 and Comparative Examples 1-3). Although, in the examples and comparative examples, the unit of the blended amount of each raw material for a liquid crystalline elastomer is “g,” the blended amounts can herein be scaled up by any reasonable factor. In other words, the unit of the blended amount of each raw material for a liquid crystalline elastomer may be replaced by “parts by weight.”

### Example 1

BH6 (100 g) as the mesogenic group-containing compound, potassium hydroxide (3.8 g), and N,N-dimethyl formamide (600 ml) as a solvent were put into a reaction container and then mixed, and 2 equivalents of propylene oxide as the alkylene oxide was added with respect to 1 mol of BH6. The resultant mixture was caused to undergo a reaction under applied pressure at 120° C. for 2 hours (addition reaction). Next, oxalic acid (3.0 g) was added to the reaction container to stop the addition reaction. Insoluble salts were removed from the reaction liquid by suction filtration. In addition, N,N-dimethyl formamide was removed from the reaction liquid by distillation under reduced pressure. Thus, a mesogenic diol A was obtained. A synthesis scheme for the mesogenic diol A is represented by formula (2). Note that the mesogenic diol A shown in formula (2) is representative and may include various structural isomers.

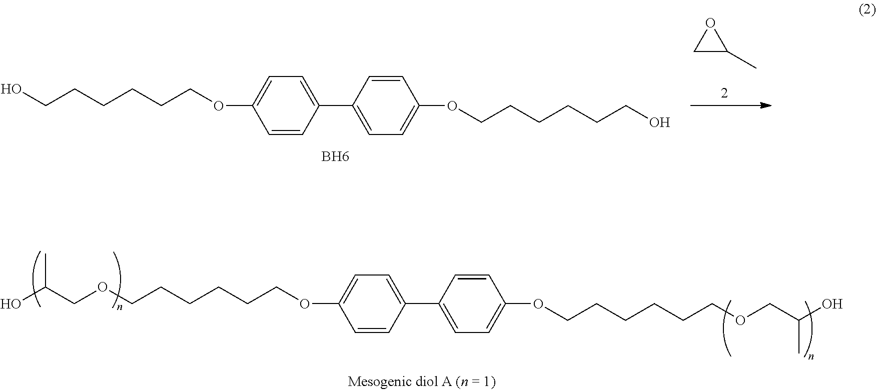

Next, the mesogenic diol A (60 g), 30 g of pyridine with respect to 1 mol of the mesogenic diol A, and N,N-dimethyl formamide (5 g) as a solvent were put into a reaction container and then mixed, and 1 equivalent of adipoyl chloride wad dropped as the dicarboxylic acid derivative with respect to 1 mol of the mesogenic diol A in 30 minutes, and the resultant mixture was stirred for 1 hour under reflux conditions (esterification reaction). Next, the reaction product was purified to obtain a liquid crystalline elastomer precursor A. A synthesis scheme for the liquid crystalline elastomer precursor A is shown in formula (3). Note that the liquid crystalline elastomer precursor A shown in formula (3) is representative and may include various structural isomers.

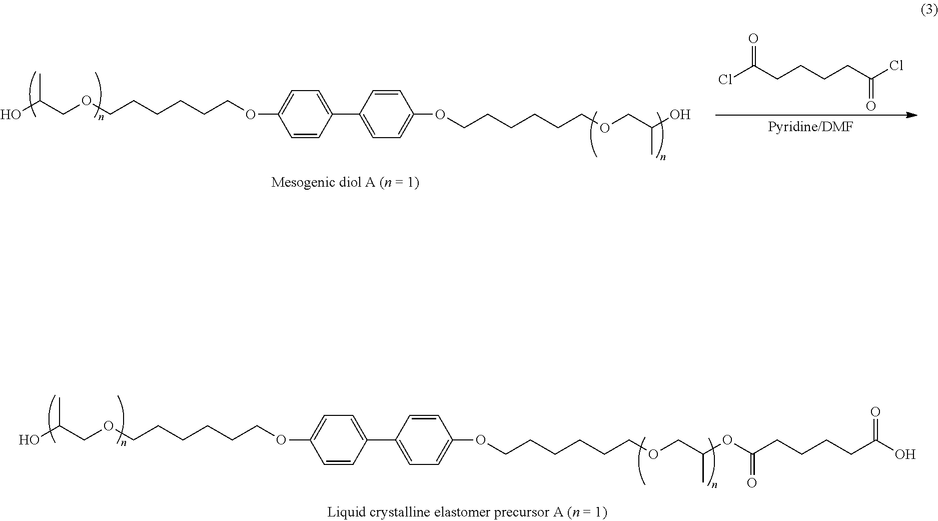

Next, the liquid crystalline elastomer precursor A (10 g), 1.1 equivalents of a blended isocyanate (a mixture of HDI isocyanurate (trade name: Sumidur (registered trademark) N3300, manufactured by Sumika Bayer Urethane Co., Ltd.) and HDI (manufactured by Nippon Polyurethane Industry Co., Ltd.), the weight ratio being 1:1) as a cross-linking agent with respect to the liquid crystalline elastomer precursor A, and 0.01 g of a catalyst (trade name: DABCO (registered trademark) T-9, manufactured by Air Products Japan K.K.) were put into a reaction container, and then stirred for 3 minutes. Next, the resultant mixture was loaded into a preheated mold, and then reacted and cured at 100° C. for 30 minutes, to obtain a half-cured liquid crystalline elastomer (pre-polymer). The pre-polymer was removed from the mold, followed by uniaxial stretching at 20° C. and a stretch ratio of 2 times. Thereafter, the pre-polymer was completely cured at 20° C. while being maintained in the stretched state, to obtain a liquid crystalline elastomer of Example 1 in which liquid crystal (mesogenic group) was aligned.

### Example 2

A mesogenic diol B, a liquid crystalline elastomer precursor B1, and a liquid crystalline elastomer of Example 2 were obtained in a manner similar to that of Example 1 in terms of raw materials and their blended amounts, and reaction conditions, stretching conditions, and curing conditions, except that four equivalents of propylene oxide was added with respect to one mol of BH6 instead of two equivalents of the oxide. Note that the mesogenic diol B, which is an intermediate, is the mesogenic diol A in formula (3) where n=2. The liquid crystalline elastomer precursor B1 is the liquid crystalline elastomer precursor A in formula (3) where n=2. These compounds may each include various structural isomers.

### Example 3

A mesogenic diol B, a liquid crystalline elastomer precursor B2, and a liquid crystalline elastomer of Example 3 were obtained in a manner similar to that of Example 1 in terms of raw materials and their blended amounts, and reaction conditions, stretching conditions, and curing conditions, except that four equivalents of propylene oxide was added with respect to one mol of BH6 instead of two equivalents of the oxide, and glutaryl chloride was added as the dicarboxylic acid derivative instead of adipoyl chloride. A synthesis scheme for the liquid crystalline elastomer precursor B2, which is an intermediate, is represented by formula (4). Note that the mesogenic diol B and liquid crystalline elastomer precursor B2 shown in formula (4) are representative and may each include various structural isomers.

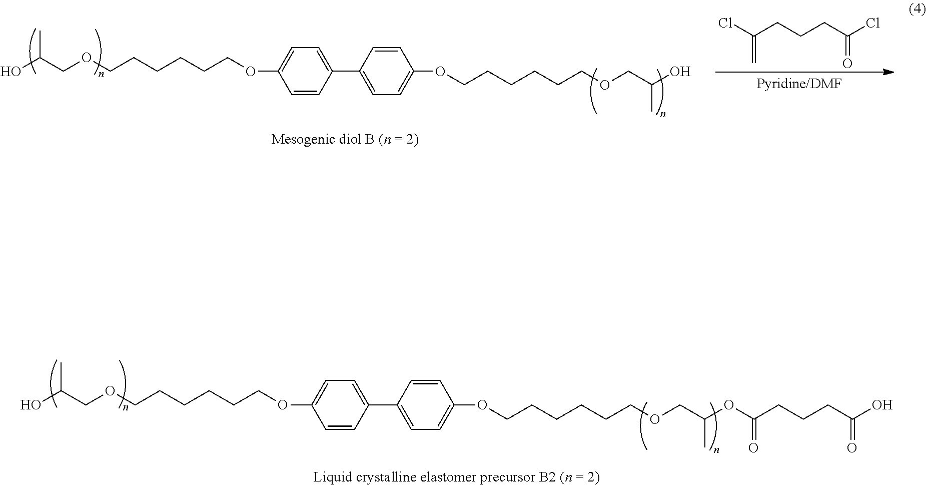

### Example 4

A mesogenic diol B, a liquid crystalline elastomer precursor B3, and a liquid crystalline elastomer of Example 4 were obtained in a manner similar to that of Example 1 in terms of raw materials and their blended amounts, and reaction conditions, stretching conditions, and curing conditions, except that four equivalents of propylene oxide was added with respect to one mol of BH6 instead of two equivalents of the oxide, and terephthaloyl chloride was added as the dicarboxylic acid derivative instead of adipoyl chloride. A synthesis scheme for the liquid crystalline elastomer precursor B3, which is an intermediate, is represented by formula (5). Note that the mesogenic diol B and liquid crystalline elastomer precursor B3 shown in formula (5) are representative and may each include various structural isomers.

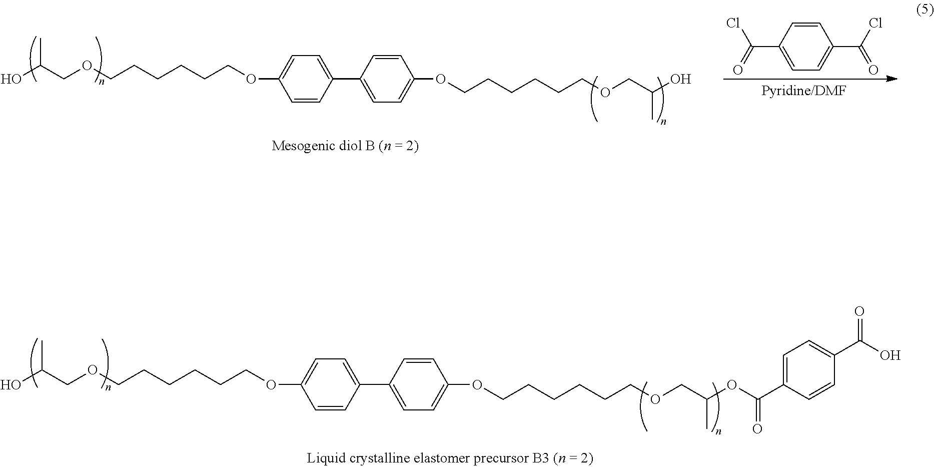

### Example 5

A mesogenic diol C, a liquid crystalline elastomer precursor C, and a liquid crystalline elastomer of Example 5 were obtained in a manner similar to that of Example 1 in terms of raw materials and their blended amounts, and reaction conditions, stretching conditions, and curing conditions, except that six equivalents of propylene oxide was added with respect to one mol of BH6 instead of two equivalents of the oxide. Note that the mesogenic diol C, which is an intermediate, is the mesogenic diol A in formula (3) where n=3. The liquid crystalline elastomer precursor C is the liquid crystalline elastomer precursor A in formula (3) where n=3. These compounds may each include various structural isomers.

### Example 6

A mesogenic diol D, a liquid crystalline elastomer precursor D, and a liquid crystalline elastomer of Example 6 were obtained in a manner similar to that of Example 1 in terms of raw materials and their blended amounts, and reaction conditions, stretching conditions, and curing conditions, except that eight equivalents of propylene oxide was added with respect to one mol of BH6 instead of two equivalents of the oxide. Note that the mesogenic diol D, which is an intermediate, is the mesogenic diol A in formula (3) where n=4. The liquid crystalline elastomer precursor D is the liquid crystalline elastomer precursor A in formula (3) where n=4. These compounds may each include various structural isomers.

### Example 7

A mesogenic diol E, a liquid crystalline elastomer precursor E, and a liquid crystalline elastomer of Example 7 were obtained in a manner similar to that of Example 1 in terms of raw materials and their blended amounts, and reaction conditions, stretching conditions, and curing conditions, except that five equivalents of butylene oxide was added as the alkylene oxide with respect to one mol of BH6 instead of two equivalents of proplylene oxide. A synthesis scheme for the mesogenic diol E, which is an intermediate, is represented by formula (6). Note that the mesogenic diol E shown in formula (6) and the liquid crystalline elastomer precursor E (not shown) obtained by cross-linking the mesogenic diol E are representative and may each include various structural isomers.

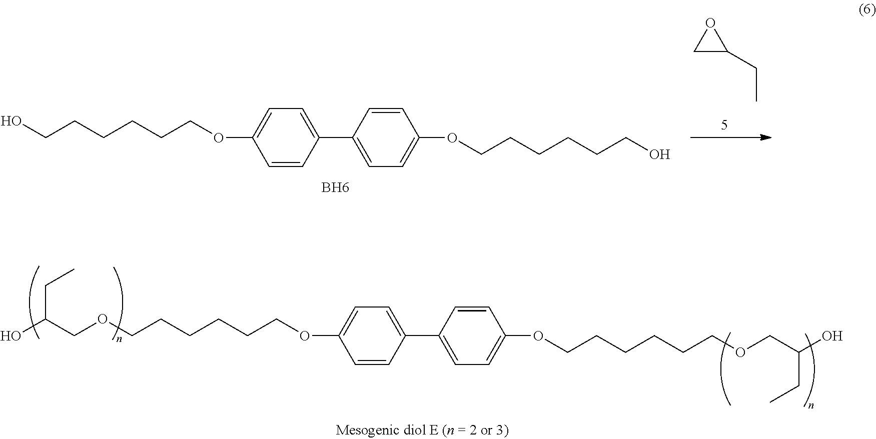

### Example 8

A mesogenic diol F, a liquid crystalline elastomer precursor F, and a liquid crystalline elastomer of Example 8 were obtained in a manner similar to that of Example 1 in terms of raw materials and their blended amounts, and reaction conditions, stretching conditions, and curing conditions, except that two equivalents of styrene oxide was added as the alkylene oxide with respect to one mol of BH6 instead of two equivalents of propylene oxide. A synthesis scheme for the mesogenic diol F, which is an intermediate, is represented by formula (7). Note that the mesogenic diol F shown in formula (7) and the liquid crystalline elastomer precursor F (not shown) obtained by cross-linking the mesogenic diol F are representative and may each include various structural isomers.

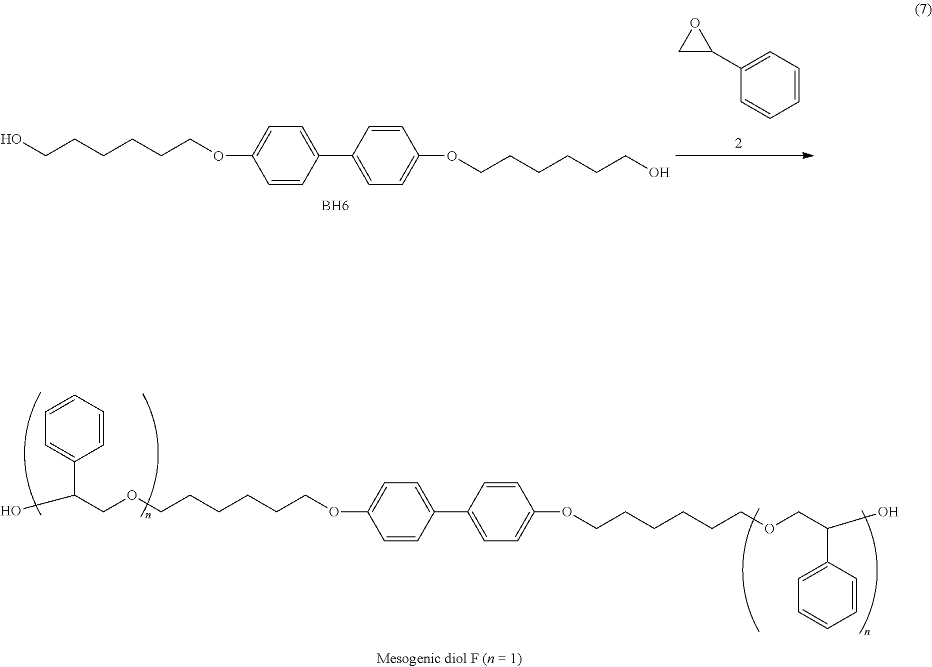

### Example 9

A mesogenic diol G, a liquid crystalline elastomer precursor G, and a liquid crystalline elastomer of Example 9 were obtained in a manner similar to that of Example 1 in terms of raw materials and their blended amounts, and reaction conditions, stretching conditions, and curing conditions, except that BHBA6 was used as the mesogenic group-containing compound, and four equivalents of propylene oxide was added with respect to one mol of BHBA6 instead of two equivalents of the oxide. A synthesis scheme for the mesogenic diol G, which is an intermediate, is represented by formula (8). Note that the mesogenic diol G shown in formula (8) and the liquid crystalline elastomer precursor G (not shown) obtained by cross-linking the mesogenic diol G are representative and may each include various structural isomers.

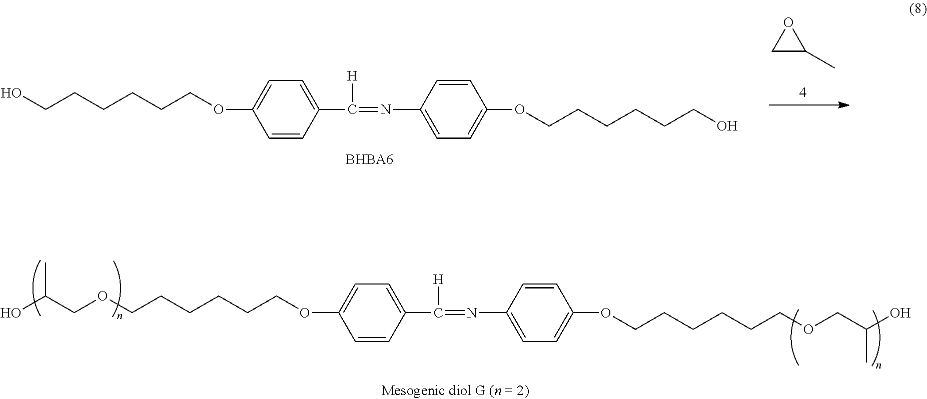

### Example 10

A mesogenic diol H, a liquid crystalline elastomer precursor H, and a liquid crystalline elastomer of Example 10 were obtained in a manner similar to that of Example 1 in terms of raw materials and their blended amounts, and reaction conditions, stretching conditions, and curing conditions, except that BA6 was used as the mesogenic group-containing compound, and four equivalents of propylene oxide was added with respect to one mol of BA6 instead of two equivalents of the oxide. A synthesis scheme for the mesogenic diol H, which is an intermediate, is represented by formula (9). Note that the mesogenic diol H shown in formula (9) and the liquid crystalline elastomer precursor H (not shown) obtained by cross-linking the mesogenic diol H are representative and may each include various structural isomers.

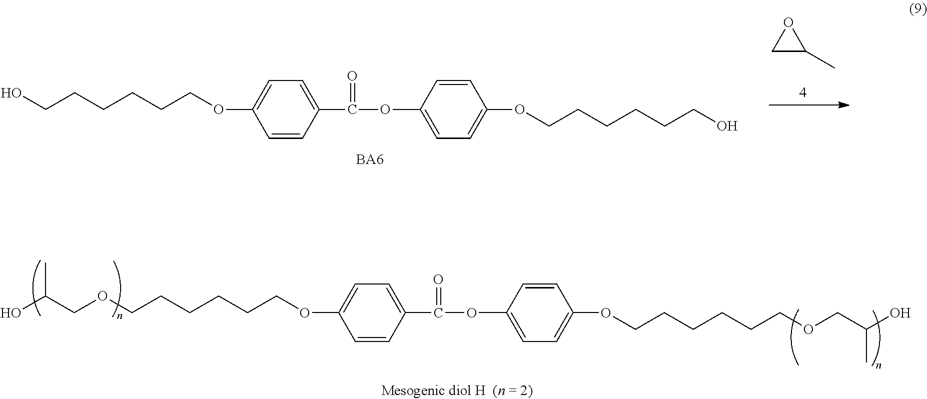

### Example 11

A mesogenic diol I, a liquid crystalline elastomer precursor I, and a liquid crystalline elastomer of Example 11 were obtained in a manner similar to that of Example 1 in terms of raw materials and their blended amounts, and reaction conditions, stretching conditions, and curing conditions, except that BH0 was used as the mesogenic group-containing compound, and two equivalents of ethylene oxide was added with respect to one mol of BH0 instead of two equivalents of propylene oxide. A synthesis scheme for the mesogenic diol I, which is an intermediate, is represented by formula (10). Note that the mesogenic diol I shown in formula (10) and the liquid crystalline elastomer precursor I (not shown) obtained by cross-linking the mesogenic diol I are representative and may each include various structural isomers.

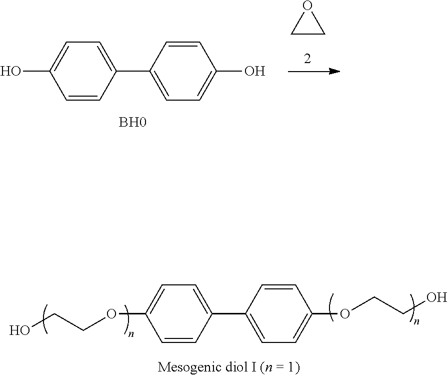

### Example 12

A mesogenic diol J, a liquid crystalline elastomer precursor J, and a liquid crystalline elastomer of Example 12 were obtained in a manner similar to that of Example 1 in terms of raw materials and their blended amounts, and reaction conditions, stretching conditions, and curing conditions, except that BH0 was used as the mesogenic group-containing compound, and four equivalents of propylene oxide was added with respect to one mol of BH0 instead of two equivalents of the oxide. A synthesis scheme for the mesogenic diol J, which is an intermediate, is represented by formula (11). Note that the mesogenic diol J shown in formula (11) and the liquid crystalline elastomer precursor J (not shown) obtained by cross-linking the mesogenic diol J are representative and may each include various structural isomers.

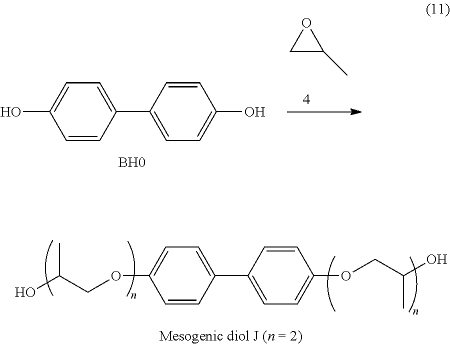

### Comparative Example 1

A liquid crystalline elastomer precursor K1 (not shown) and a liquid crystalline elastomer of Comparative Example 1 were obtained in a manner similar to that of Example 1 in terms of raw materials and their blended amounts, and reaction conditions, stretching conditions, and curing conditions, except that adipoyl chloride, which is a dicarboxylic acid derivative, was reacted with BH6, which is a mesogenic group-containing compound, instead of the mesogenic diol A, without adding an oxide to the mesogenic group-containing compound.

### Comparative Example 2

A liquid crystalline elastomer precursor K2 (not shown) and a liquid crystalline elastomer of Comparative Example 2 were obtained in a manner similar to that of Example 1 in terms of raw materials and their blended amounts, and reaction conditions, stretching conditions, and curing conditions, except that glutaryl chloride, which is a dicarboxylic acid derivative, was reacted with BH6, which is a mesogenic group-containing compound, instead of the mesogenic diol A, without adding an oxide to the mesogenic group-containing compound.

### Comparative Example 3

A liquid crystalline elastomer precursor K3 (not shown) and a liquid crystalline elastomer of Comparative Example 3 were obtained in a manner similar to that of Example 1 in terms of raw materials and their blended amounts, and reaction conditions, stretching conditions, and curing conditions, except that terephthaloyl chloride, which is a dicarboxylic acid derivative, was reacted with BH6, which is a mesogenic group-containing compound, instead of the mesogenic diol A, without adding an oxide to the mesogenic group-containing compound.

### Characteristics of Liquid Crystalline Elastomer

Characteristics (physical properties) of the liquid crystalline elastomers of Examples 1-12 and Comparative Examples 1-3 as thermoresponsive materials were investigated by measuring the phase transition temperatures (Ti), liquid crystallinity, and expansion/contraction ratio. Methods and conditions for measurement of the properties will now be described.

### Phase Transition Temperature (Ti)

The phase transition temperature (Ti) of each sample was measured using a differential scanning calorimeter (DSC) (X-DSC 7000, manufactured by Hitachi High-Tech Science Corporation). During the measurement, the temperature was increased at a rate of 20° C./min.

### Liquid Crystallinity

The presence or absence of the liquid crystallinity of each sample was verified by observation using a polarizing microscope (LV-100POL, manufactured by Nikon Corporation). The presence or absence of the liquid crystallinity of each sample was further verified from the results of measurement using the above differential scanning calorimeter (DSC).

### Expansion/Contraction Ratio

Sizes in an alignment direction of each sample in the liquid crystalline phase and the isotropic phase were measured using a scale. An expansion/contraction ratio was calculated using the following expression.

Expansion/contraction ratio (%)=(L₁-L₂)×100/L₂

L₁: The length (mm) in the alignment direction of the sample in the liquid crystalline phase

L₂: The length (mm) in the alignment direction of the sample in the isotrophic phase

The molecular structures and measurement results of the sample liquid crystalline elastomer precursors are shown in Tables 1 and 2. Note that, concerning the molecular structures of the liquid crystalline elastomer precursors in the tables, (C₆H₄) or (C₆H₅) each represent a benzene ring.

TABLE 1

| | | Example1 | Example2 | Example3 | Example4 | Example5 |
|---|---|---|---|---|---|---|
| Structure | X | Single bond | Single bond | Single bond | Single bond | Single bond |
| of liquid | A₁, A₂ | —(C₆H₄)— | —(C₆H₄)— | —(C₆H₄)— | —(C₆H₄)— | —(C₆H₄)— |
| crystalline | Y₁, Y₂ | —O— | —O— | —O— | —O— | —O— |
| elastomer | B₁, B₂ | —(CH₂)₆— | —(CH₂)₆— | —(CH₂)₆— | —(CH₂)₆— | —(CH₂)₆— |
| precursor | C₁, C₂ | * —O(C₃H₆)— | * —(O(C₃H₆))₂— | * —(O(C₃H₆))₂— | * —(O(C₃H₆))₂— | * —(O(C₃H₆))₃— |
| represented | | | | | | |
| by general | D₁ | —O—CO— | —O—CO— | —O—CO— | —O—CO— | —O—CO— |
| formula (1) | D₂ | Single bond | Single bond | Single bond | Single bond | Single bond |
| | E₁ | —(CH₂)₄CO— | —(CH₂)₄CO— | —(CH₂)₃CO— | —(C₆H₄)CO— | —(CH₂)₄CO— |
| | E₂ | Single bond | Single bond | Single bond | Single bond | Single bond |
| | Z₁, Z₂ | —OH | —OH | —OH | —OH | —OH |

| | | | | | |
|---|---|---|---|---|---|
| Isocyanate compound | Blended | Blended | Blended | Blended | Blended |
| | isocyanate | isocyanate | isocyanate | isocyanate | isocyanate |
| Ti (° C.) of liquid | 60 | 37 | 35 | 55 | 21 |
| crystalline elastomer | | | | | |
| precursor | | | | | |
| Ti (° C.) of liquid | 40 | 22 | 20 | 41 | 2 |
| crystalline elastomer | | | | | |
| Crystallinity of liquid | Yes | Yes | Yes | Yes | Yes |
| crystalline elastomer | | | | | |
| Expansion/contraction | 20 | 10 | — | — | — |
| ratio (%) of liquid | | | | | |
| crystalline elastomer | | | | | |

| | | Example6 | Example7 | Example8 |
|---|---|---|---|---|
| Structure | X | Single bond | Single bond | Single bond |
| of liquid | A₁, A₂ | —(C₆H₄)— | —(C₆H₄)— | —(C₆H₄)— |
| crystalline | Y₁, Y₂ | —O— | —O— | —O— |
| elastomer | B₁, B₂ | —(CH₂)₆— | —(CH₂)₆— | —(CH₂)₆— |
| precursor | C₁, C₂ | * —(O(C₃H₆))₄— | ** —(O(C₄H₈))₂— | —O(C₂H₃(C₆H₅))— |
| represented | | | or | |
| by general | | | —(O(C₄H₈))₃— | |
| formula (1) | | | | |
| | D₁ | —O—CO— | —O—CO— | —O—CO— |
| | D₂ | Single bond | Single bond | Single bond |
| | E₁ | —(CH₂)₄CO— | —(CH₂)₄CO— | —(CH₂)₄CO— |
| | E₂ | Single bond | Single bond | Single bond |
| | Z₁, Z₂ | —OH | —OH | —OH |

| | | | |
|---|---|---|---|
| Isocyanate compound | Blended | Blended | Blended |
| | isocyanate | isocyanate | isocyanate |
| Ti (° C.) of liquid | 12 | 35 | 82 |
| crystalline elastomer | | | |
| precursor | | | |
| Ti (° C.) of liquid | −8 | 18 | 55 |
| crystalline elastomer | | | |
| Crystallinity of liquid | Yes | Yes | Yes |
| crystalline elastomer | | | |
| Expansion/contraction | — | — | — |
| ratio (%) of liquid | | | |
| crystalline elastomer | | | |

* C₃H₆ represents CH₂—CH(CH₃).
** C₄H₈ represents CH₂—CH(C₂H₅).

TABLE 2

| | | Example9 | Example10 | Example11 | Example12 |
|---|---|---|---|---|---|
| Structure | X | —CH═N— | —CO—O— | Single bond | Single bond |
| of liquid | A₁, A₂ | —(C₆H₄)— | —(C₆H₄)— | —(C₆H₄)— | —(C₆H₄)— |
| crystalline | Y₁, Y₂ | —O— | —O— | Single bond | Single bond |
| elastomer | B₁, B₂ | —(CH₂)₆— | —(CH₂)₆— | Single bond | Single bond |
| precursor | C₁, C₂ | * —(O(C₃H₆))₂— | * —(O(C₃H₆))₂— | —O(CH₂)₂— | * —(O(C₃H₆))₂— |
| represented | | | | | |
| by general | D₁ | —O—CO— | —O—CO— | —O—CO— | —O—CO— |
| formula (1) | D₂ | Single bond | Single bond | Single bond | Single bond |
| | E₁ | —(CH₂)₄CO— | —(CH₂)₄CO— | —(CH₂)₄CO— | —(CH₂)₄CO— |
| | E₂ | Single bond | Single bond | Single bond | Single bond |
| | Z₁, Z₂ | —OH | —OH | —OH | —OH |

| | | | | |
|---|---|---|---|---|
| Isocyanate compound | Blended | Blended | Blended | Blended |
| | isocyanate | isocyanate | isocyanate | isocyanate |
| Ti (° C.) of liquid | 30 | 36 | 118 | 32 |
| crystalline elastomer | | | | |
| precursor | | | | |
| Ti (° C.) of liquid | 11 | 16 | 93 | 11 |
| crystalline elastomer | | | | |
| Crystallinity of liquid | Yes | Yes | Yes | Yes |
| crystalline elastomer | | | | |
| Expansion/contraction | — | — | — | — |
| ratio (%) of liquid | | | | |
| crystalline elastomer | | | | |

| | | Comparative | Comparative | Comparative |
|---|---|---|---|---|
| | | example1 | Example2 | Example3 |
| Structure | X | Single bond | Single bond | Single bond |
| of liquid | A₁, A₂ | —(C₆H₄)— | —(C₆H₄)— | —(C₆H₄)— |
| crystalline | Y₁, Y₂ | —O— | —O— | —O— |
| elastomer | B₁, B₂ | —(CH₂)₆— | —(CH₂)₆— | —(CH₂)₆— |
| precursor | C₁, C₂ | Single bond | Single bond | Single bond |
| represented | | | | |
| by general | D₁ | —O—CO— | —O—CO— | —O—CO— |
| formula (1) | D₂ | Single bond | Single bond | Single bond |
| | E₁ | —(CH₂)₄CO— | —(CH₂)₃CO— | —(C₆H₄)CO— |
| | E₂ | Single bond | Single bond | Single bond |
| | Z₁, Z₂ | —OH | —OH | —OH |

| | | | |
|---|---|---|---|
| Isocyanate compound | Blended | Blended | Blended |
| | isocyanate | isocyanate | isocyanate |
| Ti (° C.) of liquid | 125 | 126 | 147 |
| crystalline elastomer | | | |
| precursor | | | |
| Ti (° C.) of liquid | 106 | 108 | 122 |
| crystalline elastomer | | | |
| Crystallinity of liquid | Yes | Yes | Yes |
| crystalline elastomer | | | |
| Expansion/contraction | — | — | — |
| ratio (%) of liquid | | | |
| crystalline elastomer | | | |

* C₃H₆ represents CH₂—CH(CH₃).

It was observed that all the liquid crystalline elastomer precursors of Examples 1-12 have a phase transition temperature (Ti) of 10-120° C. It was also observed that all the liquid crystalline elastomers of Examples 1-12 obtained by cross-linking these precursors have a phase transition temperature (Ti) of −10 to 100° C., which is lower than those of their precursors by approximately 20° C., and have liquid crystallinity. Each example will now be discussed.

The liquid crystalline elastomer of Example 1 had an expansion/contraction ratio of 20%. The liquid crystalline elastomer of Example 2 had an expansion/contraction ratio of 10%. The reason why Example 2 had a smaller expansion/contraction ratio than that of Example 1 may be that as the amount of the oxide attached to the mesogenic group-containing compound increases, the proportion of the mesogenic group in the liquid crystalline elastomer decreases, so that the displace amount of the mesogenic group decreases relative to the entire liquid crystalline elastomer. Thus, it is suggested that the expansion/contraction ratio can be adjusted by changing the amount of the oxide attached to the mesogenic group-containing compound. Comparison of Examples 1, 2, 5, and 6 shows that as the amount of the oxide attached to the mesogenic group-containing compound increases, the phase transition temperature (Ti) of the liquid crystalline elastomer tends to decrease. Comparison of Examples 2-4 shows that the phase transition temperature (Ti) of the liquid crystalline elastomer in which the dicarboxylic acid attached to the mesogenic diol A does not have a benzene ring tends to be lower than when the dicarboxylic acid attached to the mesogenic diol A has a benzene ring. Similarly, comparison of Examples 2 and 8 shows that the phase transition temperature (Ti) of the liquid crystalline elastomer in which the structure of the oxide attached to the mesogenic group-containing compound does not have a benzene ring tends to be lower than when the structure of the oxide attached to the mesogenic group-containing compound has a benzene ring. Comparison of Examples 2 and 12 shows that the length of the carbon chain in the mesogenic group-containing compound does not have much influence on the phase transition temperature (Ti) of the liquid crystalline elastomer. However, comparison of Examples 11 and 12 shows that the phase transition temperature (Ti) of the liquid crystalline elastomer in which the structure of the oxide attached to the mesogenic group-containing compound has a side chain tends to be significantly lower than when the structure of the oxide attached to the mesogenic group-containing compound does not have a side chain. In addition, comparison of Examples 2 and 7 shows that the phase transition temperature (Ti) of the liquid crystalline elastomer tends to decrease irrespective of the carbon chain length of the side chain in the structure of the oxide attached to the mesogenic group-containing compound. Furthermore, comparison of Examples 2, 9, and 10 shows that the structure between the two benzene rings in the mesogenic group backbone does not have much influence on the phase transition temperature (Ti) of the liquid crystalline elastomer.

Thus, it was demonstrated that the liquid crystalline elastomers of Examples 1-12 undergo phase transition between the liquid crystalline phase and the isotropic phase at a relatively low phase transition temperature (Ti). In particular, the liquid crystalline elastomers of Examples 1 and 4 undergo phase transition at a temperature close to human body temperature. Therefore, the liquid crystalline elastomer of the present invention changes its state due to displacement of the matrix thereof in a relatively low temperature region including room temperature or human body temperature, so that the liquid crystalline elastomer changes its structure, which allows adjustment of the phase transition temperature (Ti). Therefore, the liquid crystalline elastomer of the present invention can be used as a thermoresponsive material that is convenient and practically useful.

In contrast to this, all the liquid crystalline elastomer precursors of Comparative Examples 1-3 had a phase transition temperature (Ti) of more than 120° C. In addition, all the liquid crystalline elastomers of Comparative Examples 1-3 obtained by cross-linking these precursors had liquid crystallinity, and a phase transition temperature (Ti) of more than 100° C. Therefore, none of the liquid crystalline elastomers of Comparative Examples 1-3 underwent phase transition or had elasticity in a practical, relatively low temperature region including room temperature or human body temperature.

### INDUSTRIAL APPLICABILITY

Thanks to their thermoresponsiveness and elasticity in a relatively low temperature region, the liquid crystalline elastomer precursor and liquid crystalline elastomer of the present invention are applicable to, for example, products that humans wear, such as clothing products and supports. The liquid crystalline elastomer of the present invention is also applicable to medical and health care fields such as artificial muscles and catheters, and industrial fields such as actuators and filters.

1. A liquid crystalline elastomer precursor comprising:
a mesogenic group to which an oxide compound is attached,
wherein
the liquid crystalline elastomer precursor has a molecular moiety excluding the mesogenic group, the molecular moiety having at least one ester bond and at least two active hydrogen groups.

2. The liquid crystalline elastomer precursor of claim 1, wherein
the liquid crystalline elastomer precursor is represented by:
Z₁-E₁-D₁-C₁-B₁-Y₁-A₁-X-A₂-Y₂-B₂-C₂-D₂-E₂-Z₂ (1)
where X is a portion of the molecular structure of the mesogenic group and represents a single bond that forms a portion of binding groups adjacent thereto, —N═N—, —CO—, —CH═N—, —CO—O—, —CH₂—, —CH═CH—, or —CO—NH—, A₁ and A₂ are the same or different and independently represent a cycloalkane having 3-8 carbon atoms, a benzene ring, naphthalene, biphenyl, or a heterocyclic compound thereof, optionally partially substituted with —Br, —Cl, or —CH₃, Y₁ and Y₂ are the same or different and independently represent a single bond that forms a portion of binding groups adjacent thereto, —O—, —CO—, —S—, —Se—, or —Te—, B₁ and B₂ are the same or different and independently represent a single bond that forms a portion of binding groups adjacent thereto, or —(CH₂)ₘ— where m represents an integer of 1-20, C₁ and C₂ are a binding group derived from the oxide compound, are the same or different, and independently represent —((CH₂H₂ₙ)O)ₚ— where n represents an integer of 2-4 and p represents an integer of 1-5, or —(((C₆H₅)C₂H₃)O)_{q}— where q represents an integer of 1-5, at least one of D₁ and D₂ represents the ester bond, E₁ and E₂ are the same or different and independently represent a single bond that forms a portion of binding groups adjacent thereto, provided that the adjacent binding groups are not the ester bond, —CO—, —(CH₂)ᵣCO— where r represents an integer of 1-8, or —(C₆H₄)CO—, Z₁ and Z₂ are a terminal group having the active hydrogen group, are the same or different, and independently represent —OH, —SH, —COOH, —CHO, or —O—CH(OH)—CH₂OH.

3. The liquid crystalline elastomer precursor of claim 2, wherein
the X represents a single bond that forms a portion of binding groups adjacent thereto, —CH═N—, or —CO—O—, the A₁ and the A₂ are the same and represent a benzene ring, the Y₁ and the Y₂ are the same and represent —O—, the B₁ and the B₂ are the same and represent a single bond that forms a portion of binding groups adjacent thereto, or —(CH₂)₆—, the C₁ and the C₂ are the same and represent —((CₙH₂ₙ)O)ₚ— where n represents an integer of 2-4 and p represents an integer of 1-4, or —((C₆H₅)C₂H₃)O—, the D₁ represents the ester bond, the D₂ represents a single bond that forms a portion of binding groups adjacent thereto, the E₁ represents —(CH₂)ᵣCO— where r represents an integer of 3 or 4, or —(C₆H₄)CO—, the E₂ represents a single bond that forms a portion of binding groups adjacent thereto, and the Z₁ and the Z₂ are the same and represent —OH.

4. A liquid crystalline elastomer comprising:
the liquid crystalline elastomer precursor of claim 1 cross-linked by a trifunctional or higher-functional isocyanate compound and/or polyol compound, wherein
the liquid crystalline elastomer has a molecular moiety excluding the mesogenic group, the molecular moiety having at least one ester bond, and
the liquid crystalline elastomer reversibly changes a state thereof between a liquid crystalline phase and isotropic phase thereof in response to a change in temperature thereof.

5. (canceled)

6. A liquid crystalline elastomer comprising:
the liquid crystalline elastomer precursor of claim 2 cross-linked by a trifunctional or higher-functional isocyanate compound and/or polyol compound,
wherein
the liquid crystalline elastomer has a molecular moiety excluding the mesogenic group, the molecular moiety having at least one ester bond, and
the liquid crystalline elastomer reversibly changes a state thereof between a liquid crystalline phase and isotropic phase thereof in response to a change in temperature thereof.

7. A liquid crystalline elastomer comprising:
the liquid crystalline elastomer precursor of claim 3 cross-linked by a trifunctional or higher-functional isocyanate compound and/or polyol compound,
wherein
the liquid crystalline elastomer has a molecular moiety excluding the mesogenic group, the molecular moiety having at least one ester bond, and
the liquid crystalline elastomer reversibly changes a state thereof between a liquid crystalline phase and isotropic phase thereof in response to a change in temperature thereof.

8. The liquid crystalline elastomer of claim 4, wherein
a phase transition temperature (Ti) that is a boundary between the liquid crystalline phase and the isotropic phase is −10 to 100° C.

9. The liquid crystalline elastomer of claim 6, wherein
a phase transition temperature (Ti) that is a boundary between the liquid crystalline phase and the isotropic phase is −10 to 100° C.

10. The liquid crystalline elastomer of claim 7, wherein
a phase transition temperature (Ti) that is a boundary between the liquid crystalline phase and the isotropic phase is −10 to 100° C.
